(19) United States
(12) Patent Application Publication
(10) Pub. No.: US 2001/0020471 A1
(43) Pub. Date: Sep. 13, 2001

(54) Respiratory anesthesia device with controlled relief valve

(76) Inventor: Sebastien Kitten, Saulx Les Chartreux (FR)

Correspondence Address: YOUNG & THOMPSON, 745 SOUTH 23RD STREET 2ND FLOOR, ARLINGTON, VA, 22202

(21) Appl. No.: 09/765,443

(22) Filed: Jan. 22, 2001

(30) Foreign Application Priority Data
Jan. 21, 2000 (FR) 0000781

(51) Int. Cl. A61M 15/00; A61M 16/00; A62B 7/00; F16K 31/02

(52) U.S. Cl. 128/203.14; 128/204.21

(57) ABSTRACT

Respiratory anesthesia device comprising a gas circuit (1) comprising an inhalation branch for conveying a mixture of anesthetic gas to connection means (24) intended to be connected to the upper airways of a patient (11); an exhalation branch (3) for conveying a gaseous mixture containing CO₂ exhaled by said patient (11); said inhalation branch (2) and said exhalation branch (3) forming a looped gas circuit (1). A line (8) supplying fresh gas, in fluidic communication with the gas circuit (1) is intended to supply said circuit (1) with fresh gas. Furthermore, a relief valve (4) with an adjustable pressure set-point value is arranged on the inhalation branch (2) or on the exhalation branch (3) and means for controlling the relief valve (4) acting on the relief valve (4) in response to the detection of a variation in flow rate and/or pressure of the fresh gas inside the fresh gas supply line (8) so as to adjust the pressure set-point of the relief valve (4).

The present invention relates to a respiratory anesthesia device comprising a device that makes it possible to reduce the time constant of the patient circuit by feedback control of the relief valve of said patient circuit.

In a way known per se, anesthesia devices conventionally operate as a closed circuit.

Specifically, these devices generally comprise devices for recovering the gas exhaled by the patient so as to rid it of the CO₂ it contains and return it to the patient during a subsequent inhalation phase.

Furthermore, anesthesia devices comprise a device that allows the patient to be ventilated by administering a gas which contains anesthetic vapors to him.

The patient consumes some of the gas inhaled and exhales the rest.

The gases exhaled by the patient are recovered, rid of the CO₂ so as to be returned to the patient. The consumption by the patient is compensated for by a top-up of fresh gas. In addition, defects of leaktightness of the circuit create a deficit of gas during ventilation and exhalation. This deficit is also compensated for by the top-up of fresh gas.

The gaseous concentrations of the mixture with which the patient is ventilated vary as claimed in the medical prescription, during anesthesia. As these concentrations change, the practitioner increases the fresh gas top-up, while excess is discharged through a specific valve.

By altering the flow rate of fresh gas, it is possible directly to increase or reduce the rate at which the concentrations in the circuit are modified. What this means is that the fresh gas consumption may be increased greatly, which incurs a significant additional cost and leads to increased pollution of the operating block with anesthetic vapors.

Anesthesia ventilators of this kind are described in documents EP-A-0761249 and EP-A-0745404.

In other words, given the diversity of situations encountered during surgical, medical or similar intervention employing respiratory anesthesia of the patient, achieved using a respiratory anesthesia device, the operator of the device, for example a doctor, a nurse or the like, is often forced to modify the specifics of the ventilation of the patient and, in particular, to vary the amount of fresh gas sent to the patient circuit, particularly to the inhalation branch of this patient circuit.

To do this, anesthesia devices operating in a closed circuit with the gases re-inhaled are supplied with a top-up of roughly constant flow rate of fresh gas and employ a relief valve to restrict the internal pressure in the patient circuit.

The relief valve (of whatever kind) is customarily arranged in the inhalation branch or in the exhalation branch of the patient circuit.

This relief valve makes it possible to avoid any inopportune rise in gas pressure in the patient circuit above a preordained set-point pressure value.

However, in the case of high gas flow rates, particularly during the concentration changes, it has been found in practice that the relief valve almost always maintained a pressure markedly higher than the nominal set-point pressure, this being on account of the pressure drops in the system as a whole.

In other words, there is no current system that takes account of the influences that the flow rate and/or pressure of fresh gas has on the residual pressure in the circuit induced by the relief valve and therefore the residual pressure in the patient's lungs.

At the current time, when the user wishes to modify the composition of the gaseous mixture present in the circuit by virtue of a large increase in the flow rate of fresh gas to a new concentration, he modifies the relief valve opening value, thus causing a pressure increase in the patient circuit.

Now, this pressure increase then leads to an increase in the pressure in the patient's lungs, which has the effect of reducing the passage of blood through the lungs and thereby of reducing the effectiveness of the variations in concentration of the gases inhaled, which is counter to the desired objective.

Furthermore, the pressure in the circuit and induced by the relief valve opening pressure serves to keep in the inflated state the accumulation member which is used for recovering the exhaled gases. This accumulation member plays a part in diluting the fresh gases in the patient circuit and its internal volume directly introduces a delay in concentration modification.

Hence, it will be readily understood that a reduction in the relief valve opening pressure makes it possible in particular to reduce the volume of the accumulation member and improve the time constant of the circuit with respect to concentration variations, whereas in fact increasing the flow rate of fresh gases produces the opposite effect on the relief valve.

This then means that increasing the flow rate of fresh gas leads to indirect effects which are counter to the desired objective.

Hence, the only solution that the operator has available to him in order to accelerate the response time of the system to concentration variations is to considerably increase the flow rate of fresh gas because a slight increase in the flow rate is not proving sufficient.

The user thus causes fresh gases and therefore halogenated products used in the anesthesia to be rejected into the atmosphere.

The object of the present invention is therefore to solve the aforementioned problems by proposing a respiratory anesthesia device capable of taking account of the value of the flow rate of fresh gases and of incorporating it into the control of the relief valve so as to make a saving on the time constant of the circuit and therefore economize on fresh gases, particularly halogenated compounds, without creating side effects in the patient which would be against the care strategy set up by the doctor.

The present invention therefore relates to a respiratory anesthesia device comprising:

a gas circuit comprising:

(a) an inhalation branch capable of conveying a mixture of anesthetic gas to connection means intended to be connected to the upper airways of a patient;

(b) an exhalation branch capable of conveying a gaseous mixture containing CO₂ exhaled by said patient;

said inhalation branch and said exhalation branch forming the looped gas circuit,

at least one line supplying fresh gas, in fluidic communication with at least part of said gas circuit, and intended to supply said gas circuit with fresh gas;

at least one relief valve with an adjustable pressure set-point value, arranged on the inhalation branch or on the exhalation branch; and

means for controlling the relief valve acting on said relief valve in response to the detection of a variation in flow rate and/or pressure of the fresh gas inside at least part of the fresh gas supply line so as to adjust the pressure set-point of said relief valve.

Depending on the situation, the device may have one or more of the following features:

the control means act in response to a variation in at least the flow rate in at least part of the supply line.

the control means comprise flow and/or pressure-measurement means arranged on the fresh gas supply line and electronic processing means electrically connected to said measurement means.

the electronic processing means act on the relief valve in response to said measurement means.

the processing means command a decrease in the pressure set-point value of the relief valve in response to the measurement means determining an increase in the flow rate of fresh gas in the supply line.

the processing means act on the relief valve via a pressure control line.

the control means comprise a venturi device arranged in the fresh gas supply line, said venturi device being pneumatically connected to a pressure equalizing device, said equalizing device being pneumatically connected to said relief valve.

said pressure equalizing device comprises at least two internal chambers separated by a moving and/or deformable partition.

Furthermore, the invention also relates to a method for controlling a device as claimed in the invention, wherein the procedure entails the steps of:

(a) determining at least one item of information about the flow rate and/or pressure of the fresh gas flowing through the supply line using the flow and/or pressure-measurement means arranged on said line;

(b) transmitting said information to said electronic processing means and processing said information;

(c) modifying or adjusting the pressure set-point value of the relief valve.

As a preference, the information processing performed in step (b) comprises integrating the flow rate value measured in step (a) and comparing this integrated value with a reference value.

Advantageously, the pressure set-point value of the relief valve is reduced when it is found that the measured flow rate of fresh gas has increased.

The invention will now be described in greater detail with the aid of the appended figures which depict two embodiments of a device according to the invention which are given by way of nonlimiting illustration.

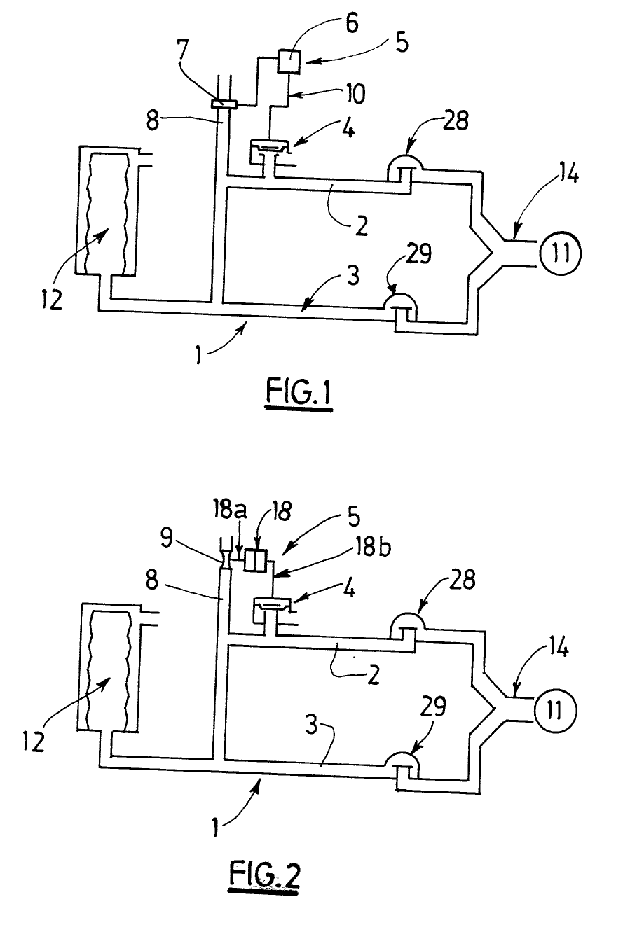

FIG. 1 depicts a diagram of a respiratory anesthesia device as claimed in the invention, which device comprises a gas circuit 1 in the form of a loop formed, on the one hand, of a inhalation branch 2 and of an exhalation branch 3.

The inhalation branch 2 conveys an anesthetic respiratory gas from a source 12 of anesthetic respiratory gas 10, in this instance an accumulation reservoir used for ventilating in machine mode, as far as connecting means 14 connected to the upper airways of a patient 11, it being possible for said connection means 14 to be a respiratory mask, intubation probes or any other similar means.

Conversely, the exhalation branch 3 allows the gases exhaled by the patient 11 during the exhalation phases to be collected, said exhaled gases being rich in carbon dioxide (CO₂) and containing anesthetic products such as halogenated products, which it is desirable to be able to recover and recycle so that they can be sent back to the patient 11 later.

Said inhalation branches 2 and exhalation branches 3 form a circuit in the form of a loop 1, also known as a closed circuit or patient circuit.

The inhalation branch 2 is equipped with a valve 4 venting to atmosphere, making it possible to relieve any overpressure in said inhalation branch, which overpressure could be harmful to the patient 11. This vent valve 4 is commonly known as a relief valve.

Furthermore, the exhalation branch 3 comprises, conventionally, purification means (not depicted) such as a purification device containing an absorbent, for example lime or the like, intended to remove most of the CO₂ contained in the gases exhaled by the patient.

Furthermore, the inhalation branch 2 and the exhalation branch 3 are equipped with non-return valves or shutters 28 and 29, respectively.

The embodiment in FIG. 1 further comprises control means of the electronic type acting in response to an increase in the flow rate of fresh gas in the line 8 to modify, if necessary and accordingly, the pressure set-point value of the relief valve 4.

More precisely, these control means comprise flow-measurement means 7, such as a flow meter, arranged in the line 8 and electrically connected to electronic processing means 6, such as a microprocessor card, so that the flow rate value measured in the line 8 can be transmitted to said processing means 6.

The processing means 6 therefore process the flow rate value and make it possible, as claimed in a predefined table, to determine the pressure set-point value of the relief valve 4.

In other words, the device of the invention makes it possible to measure the flow rate of fresh gas and to integrate the measured value of the flow rate in order to modify the pressure set-point applied to the relief valve 4 of the circuit 1.

Furthermore, FIG. 2 depicts a second embodiment of an anesthesia device as claimed in the invention, which is similar to FIG. 1 except for the fact that, in this instance, the relief valve 4 is controlled via a device of the pneumatic type.

More specifically, this device of pneumatic type comprises a venturi device 9 arranged on the fresh gas passage inside the supply line 8, which venturi device 9 is pneumatically connected, that is to say is placed in fluidic communication, via a pneumatic line 18a, with a pressure equalizing device 18 comprising two internal chambers separated by a moving or deformable partition.

The equalizing device 18 is itself connected pneumatically, via an equalizing line 18b, to said relief valve 4.

If the flow rate of fresh gas in the line 8 increases, a depression is created, through a venturi effect, above the shutter of the relief valve 4, so as to reduce its resistance; this depression being relayed by the pressure equalizing device 18.

The present invention is to reduce the response time of the valve 4 to a change in concentration set-point of the mixture by electronic or mechanical feedback control of the pressure that operates the valve 4.

Furthermore, in order to provide even more effective control over the pressure in the ventilator circuit 1, it is also possible to incorporate into said ventilator, a device for controlling the filling of the accumulation volume 12 of the circuit 1.

Specifically, it may sometimes prove beneficial to optimize the rate of filling of the accumulation reservoir 12 so as to just compensate for the consumption by the patient and any losses from the circuit, because if a leak occurs in the circuit, the amount of gas with which the patient is ventilated upon each respiratory cycle is liable to decrease substantially and must therefore quickly be detected.

To do this, a measurement of the rate of flow leaving the circuit via the overflow of the accumulation reservoir 12, of the rate of flow entering and leaving the accumulation reservoir and/or of the pressure inside the accumulation reservoir at the end of the exhalation phase is/are preferably taken.

All of the measurements are then processed by information processing software and information is given to the user when the accumulation reservoir does not reach a minimum filling threshold before the start of each inhalation phase.

Likewise, information is given when the flow rate at the outlet of the overflow becomes too high by comparison with the set fresh gas flow rate and can be correlated with the concentration measurements taken in the fresh gases and in the inhalation or exhalation branches or with their differences, so as to eliminate instances in which the user is deliberately setting a high fresh gas flow rate in order to obtain a quick change in concentration in his circuit.

1. A respiratory anesthesia device comprising:
a gas circuit (1) comprising:
(a) an inhalation branch (2) capable of conveying a mixture of anesthetic gas to connection means (24) intended to be connected to the upper airways of a patient (11);
(b) an exhalation branch (3) capable of conveying a gaseous mixture containing CO₂ exhaled by said patient (11);
said inhalation branch (2) and said exhalation branch (3) forming the looped gas circuit (1),
at least one line (8) supplying fresh gas, in fluidic communication with at least part of said gas circuit (1), and intended to supply said gas circuit (1) with fresh gas;
at least one relief valve (4) with an adjustable pressure set-point value, arranged on the inhalation branch (2) or on the exhalation branch (3); and
means for controlling the relief valve (4) acting on said relief valve (4) in response to the detection of a variation in flow rate and/or pressure of the fresh gas inside at least part of the fresh gas supply line (8) so as to adjust the pressure set-point of said relief valve (4).

2. The device as claimed in claim 1, wherein the control means act in response to a variation in at least the flow rate in at least part of the supply line (8).

3. The device as claimed in either of claims 1 and 2, wherein the control means comprise flow and/or pressure-measurement means (7) arranged on the fresh gas supply line (8) and electronic processing means (6) electrically connected to said measurement means (7).

4. The device as claimed in one of claims 1 to 3, wherein the electronic processing means (6) act on the relief valve (4) in response to said measurement means (7).

5. The device as claimed in one of claims 1 to 4, wherein the processing means (6) command a decrease in the pressure set-point value of the relief valve (4) in response to the measurement means (7) determining an increase in the flow rate of fresh gas in the supply line (8).

6. The device as claimed in one of claims 1 to 5, wherein the processing means (6) act on the relief valve (4) via a pressure control line (10).

7. The device as claimed in either of claims 1 and 2, wherein the control means comprise a venturi device (9) arranged in the fresh gas supply line (8), said venturi device (9) being pneumatically connected (18a) to a pressure equalizing device (18), said equalizing device being pneumatically connected (18b) to said relief valve (4).

8. The device as claimed in one of claims 1, 2 and 7, wherein said pressure equalizing device (18) comprises at least two internal chambers separated by a moving and/or deformable partition.

9. A method for controlling the device as claimed in one of claims 1 to 6, wherein the procedure entails the steps of:
(a) determining at least one item of information about the flow rate and/or pressure of the fresh gas flowing through the supply line (8) using the flow and/or pressure-measurement means (7) arranged on said line (8);
(b) transmitting said information to said electronic processing means (6) and processing said information;
(c) modifying or adjusting the pressure set-point value of the relief valve (4).

10. The method as claimed in claim 9, wherein the information processing performed in step (b) comprises integrating the flow rate value measured in step (a) and comparing this integrated value with a reference value.

11. The method as claimed in either of claims 9 and 10, wherein the pressure set-point value of the relief valve (4) is reduced when it is found that the measured flow rate of fresh gas has increased.
